(19) United States
(12) Patent Application Publication
(10) Pub. No.: US 2021/0145010 A1
(43) Pub. Date: May 20, 2021

(54) Biocompatible siloxanes for formulation of microorganisms

(71) Applicant: Evonik Operations GmbH, Essen (DE)

(72) Inventors: René Hänsel, Dorsten (DE); Katja Skrabania, Essen (DE); Jochen Kleinen, Heinsberg (DE); Michael Ferenz, Essen (DE)

(73) Assignee: Evonik Operations GmbH, Essen (DE)

(21) Appl. No.: 15/733,753

(22) PCT Filed: Apr. 12, 2019
(86) PCT No.: PCT/EP2019/059432

§ 371 Date: Oct. 16, 2020

(30) Foreign Application Priority Data
Apr. 19, 2018 (EP) 18168183.4

(51) Int. Cl. A01N 63/38; A01N 63/40

(52) U.S. Cl. CPC A01N 63/38; A01N 63/40

(57) ABSTRACT

Compositions containing at least one siloxane and at least one active microbiological ingredient are useful for the treatment of plants, of seeds or of soils, as biostimulant or as probiotic food supplement or animal feed additive, or as probiotic medicament. In addition, the siloxane improves the storage stability of an active microbiological ingredient.

The present invention provides compositions comprising at least one siloxane and at least one active microbiological ingredient, processes for production thereof, for the use thereof for the treatment of plants, seed or soils, for the use thereof as biostimulant or for the use thereof as probiotic food supplement or animal feed additive, and compositions for use as probiotic medicament, and also for the use of the siloxane for improvement of the storage stability of an active microbiological ingredient.

In agriculture, microorganisms are used for a multitude of beneficial applications, for example for biological plant protection, for biological plant fortification or for biological soil improvement. In addition, compositions comprising living microorganisms are also used for the treatment of seed. The field of use is thus especially agriculture and forestry including horticulture and pomiculture, and the growing of ornamentals and the growing and care of lawns. In addition, compositions comprising living microorganisms are also employed as probiotics in foods and animal feeds or as probiotic medicaments.

Biological plant protection products—also referred to as biopesticides—are increasingly being used in agriculture since they help to replace or reduce the use of chemical pesticides, and thus reduce residues of chemical pesticides in foods. In the event of resistances of plant pathogens and pests to chemical pesticides, biological plant protection products are alternatives. The use of biological plant protection products is increasingly being demanded by current environmental legislation, since they make use of natural regulation mechanisms that have evolved over the course of evolution, and hence conserve the environment. Biological plant protection products find use, for example, as fungicides, insecticides, nematicides or herbicides and are being used for preventative treatment or curative control of plant pathogens and pests. Active biological ingredients are specified, for example, in The Manual of Biocontrol Agents, 2001, The British Crop Protection Council.

According to Article 2 (1) of REGULATION (EC) No 1107/2009 OF THE EUROPEAN PARLIAMENT AND OF THE COUNCIL of 21 Oct. 2009 concerning the placing of plant protection products on the market and repealing Council Directives 79/117/EEC and 91/414/EEC, plant protection products refer to products, in the form in which they are supplied to the user, consisting of or containing active substances, safeners or synergists, and intended for one of the following uses:
a) protecting plants or plant products against all harmful organisms or preventing the action of such organisms, unless the main purpose of these products is considered to be for reasons of hygiene rather than for the protection of plants or plant products;
b) influencing the life processes of plants, such as substances influencing their growth, other than as a nutrient;
c) preserving plant products, in so far as such substances or products are not subject to special Community provisions on preservatives;
d) destroying undesired plants or parts of plants, except algae unless the products are applied on soil or water to protect plants;
e) checking or preventing undesired growth of plants, except algae unless the products are applied on soil or water to protect plants.

The present invention is preferably based on the abovementioned definition of the term “plant protection products”.

According to the provisional definition of the European Biostimulants Industry Council (EBIC), biostimulants contain substance(s) and/or micro-organisms whose function when applied to plants or the rhizosphere is to stimulate natural processes to enhance/benefit nutrient uptake, nutrient efficiency, tolerance to abiotic stress, and crop quality (http://www.biostimulants.eu). For example, the microorganisms Trichoderma spp., Pythium oligandrum, Bacillus spp., Pseudomonas spp. and Streptomyces spp. can cause reactions in plants that lead to elevated resistance to pathogens or other stress factors, such as drought, poor nutrient supply, unfavourable pH values and/or high salt content in the soil. The microorganisms Trichoderma spp., Penicillium bilaii, Azotobacter spp., Azotomonas spp., Azospirillum spp. and Rhizobium spp. can lead, for example, to an improvement in nutrient availability in the soil or directly at the plant roots.

Barriers to broad use of active microbiological ingredients for biological plant protection, for biological plant fortification or for biological soil improvement have to date been their lower efficacy compared to many chemical products. This lower efficacy is based, for example, on inadequate survival capacity of the microorganisms in the formulation during storage. In application, possibly too little active ingredient reaches the target locus on the plant or in the soil, where it may be rapidly degraded by environmental effects. However, these adverse aspects can be improved by a suitable formulation or by the use of adjuvants.

The biological plant protection product based on microorganisms as active constituent and the biostimulants are typically diluted in water in the form of a formulation prior to use. These formulations may, for example, be solid formulations, such as wettable powders (WP) or water-dispersible granules (WG), but also liquid formulations such as oil dispersions (OD), suspension concentrates (SC) or dispersion concentrates (DC).

The formulation brings the microorganisms into a form in which they can be handled, such that they can be distributed and applied in water. Since many microorganisms such as some genera of fungal conidia are water-repellent, a particular task of the formulation is to make them water-compatible. Moreover, the formulation should also assure the survival capacity of the microorganisms during transport and storage. The formulation is also to ensure that application can be effected by means of spray equipment; aggregation of the microorganisms is to be avoided in order that blockage of nozzles can be ruled out. The formulation is also to contain those substances that assure the dispersion and distribution of microorganisms in the water, and facilitate the application of the spray liquor to the plants or the soil.

In practice, formulations of chemical and biological plant protection products are diluted in water by the user prior to use. For this purpose, the plant protection products are typically added to a tank with water as ingredient and distributed in what is called the spray liquor while stirring. This spray liquor is a ready-to-use dilution of the plant protection products. It is typically sprayed by means of a nozzle onto the plants or the soil in a defined dosage. The spray droplets are to be very well distributed on the plant or the soil in order that an optimal effect is assured.

For improvement of the biological efficacy (also referred to as effectiveness) of chemical plant protection products, it is standard practice to use what are called adjuvants, also referred to as additives. Adjuvants are typically added to the aqueous spray liquor shortly before deployment and spray application as tankmix additive or integrated directly into plant protection product formulations. The adjuvants are typically added to the spray liquor in concentrations of 0.001% by volume to 1% by volume. The adjuvants reduce the surface tension of water and ensure improved adhesion and wetting of the spray droplets on the hydrophobic leaves of the plant, and hence homogeneous distribution of the plant protection product over a wide area. They also improve the penetration and distribution of the active constituents of the spray liquor into the soil. This increases biological efficacy. Adjuvants can likewise also improve the efficacy of microbiological plant protection products and, depending on the nature of the formulation, be used as dispersant, emulsifier and wetting agent. However, they may potentially be cytotoxic to living microorganisms and have to date only been used rarely for formulations of living microorganisms.

The Pesticides Safety Directorate (PSD, the executive branch of the Health and Safety Executive (HSE), a non-governmental public organization in Great Britain) defines an adjuvant as a substance other than water which is not itself pesticidally active but increases the effectiveness of a pesticide (http://www.hse.gov.uk/pesticides/topics/pesticide-approvals/pesticides-registration/applicant-guide/the-applicant-guide-adjuvan.htm). It refers here to REGULATION (EC) No 1107/2009 OF THE EUROPEAN PARLIAMENT AND OF THE COUNCIL of 21 Oct. 2009 concerning the placing of plant protection products on the market and repealing Council Directives 79/117/EEC and 91/414/EEC, Article 2 (3)(d). According to this, substances or preparations which consist of co-formulants or preparations containing one or more co-formulants, in the form in which they are supplied to the user and placed on the market to be mixed by the user with a plant protection product and which enhance its effectiveness or other pesticidal properties, are referred to as ‘adjuvants’. The terms “additives” or “adjuvants” are used synonymously in the present disclosure. Adjuvants used are frequently synthetic surfactants, for example ethoxylated alcohols, nonylphenol ethoxylates or alkyl polyglycosides. The use of water-soluble hydrophilic polyglyceryl esters as adjuvants in crop protection formulations is likewise known. In addition, trisiloxane surfactants are frequently used as adjuvants. These trisiloxane surfactants reduce the static surface tension of spray liquors or water to a significantly higher degree than purely organic surfactants. Trisiloxane surfactants have the general structure Me₃SiO—SiMeR-OSiMe₃ where the R radical is a polyether radical.

The prior art formulations have multiple disadvantages. It is generally the case for all formulations of microbiological plant protection products that the microorganisms present lose viability and/or germinability with time. The formulations frequently have to be stored at temperatures below 10° C. in order to assure acceptable viability and/or germinability at least for a few weeks. Solid formulations such as WP and WG formulations additionally have the disadvantage that there is the risk of inhalation to the user when measuring-out and mixing the concentrated powder or granules.

Moreover, solid formulations that are dispersed in water frequently exhibit reduced wetting of hydrophobic surfaces. Liquid formulations such as OD, SC and DC formulations, in addition to reduced viability and/or germinability of the microorganisms present, show the further disadvantage that it is generally necessary to add further additives such as dispersants, emulsifiers, and also surface-active substances (called “surfactants”), which lower the surface tension of water to such an extent that good wetting of leaves and of the soil can be assured. This leads to complex formulations, and any of these constituents can potentially have adverse effects on the viability and/or germinability of the microorganisms.

WO 2012/163322 A1 discloses a liquid preparation for biological plant protection, comprising a suspension of an active microorganism or a mixture of two or more active microorganisms or organs of microorganisms and a polyether-modified trisiloxane. Also described is the production of a dispersion concentrate (DC) from a polyether-modified trisiloxane and microorganisms. It is found that hydrophobic microorganisms can be effectively dispersed in polyether-modified trisiloxane. However, a disadvantage is the modest storability of microscopic fungi and fungal organs, for example fungal spores. It is also found that the polyether-modified trisiloxanes have low hydrolysis stability in an aqueous environment, which impairs the storability of concentrates, but also of spray liquors. In addition, viability and/or germinability of the microorganisms on storage in the spray liquor is reduced. This is important especially when work is suspended for a day and the spray liquor is stored overnight.

WO 2016/050726 A1 discloses liquid compositions comprising spores of a spore-forming fungus, a polyether-modified trisiloxane and a fumed silica or precipitated silica, where the compositions are essentially free of water. It is stated that the use of silicas slows the sedimentation of the spores.

WO 2017/116837 A1 discloses a nonaqueous composition comprising microbial spores, protectants and dispersants. Trisiloxanes are described as possible constituents of the composition. The compositions are intended to improve the viability and/or stability of microbial spores.

There is still a need to provide compositions that have distinct advantages over the prior art. More particularly, there is still a need for compounds for the production of compositions comprising active microbiological ingredients, for example fungal spores or fungal conidia.

The problem addressed by the present invention was that of overcoming at least one disadvantage of the state of the art.

A particular problem addressed was that of providing compositions that contain active microbiological ingredients and show improved handling and storability compared to the prior art, especially have improved hydrolysis stability and/or elevated viability and/or germinability of the active microbial ingredients present, for example bacteria, fungi and viruses, and hence show elevated biological efficacy compared to the prior art. Thus, a particular problem addressed was that of conserving biological efficacy and/or bioavailability over a longer period compared to the prior art.

It has been found that, surprisingly, compositions that include, as well as an active microbial ingredient, specific siloxanes as described in the claims overcome at least one disadvantage of the prior art.

More particularly, it has been found that compositions which comprise, as well as active microbial ingredients, the specific siloxanes as described in the claims lead to improved storability over the prior art, especially to improved viability and/or germinability of the active microbial ingredient present. This is to be observed not just in essentially anhydrous compositions but also in water-diluted compositions, for example in spray liquors. A further advantage of the compositions according to the invention is that the specific siloxanes present therein, after being applied to the substrate or the plant, retain the moisture for much longer than prior art compositions. They thus provide a moisture reservoir for the microorganisms and thus improve the viability and/or germinability of the microorganisms. A further advantage of the siloxanes is their broad usability.

The object of the present invention is therefore achieved by the subject-matter of the independent claims. Advantageous configurations of the invention are specified in the subsidiary claims, the examples and the description.

The inventive composition, the inventive process and the inventive use of the compositions and/or the process products are described by way of example hereinafter without any intention that the invention be restricted to these illustrative embodiments. Where ranges, general formulae or classes of compounds are specified hereinafter, these are intended to encompass not only the corresponding ranges or groups of compounds which are explicitly mentioned but also all subranges and subgroups of compounds which can be obtained by leaving out individual values (ranges) or compounds. Any embodiment that can be obtained by combination of regions/subregions and/or groups/subgroups, for example by combinations of inventive, essential, optional, preferred, preferential or preferably selected, further preferred, even further preferred, particularly preferred or especially preferred regions/subregions and/or groups/subgroups, forms a full part of the disclosure content of the present invention and is considered to be explicitly, directly and unambiguously disclosed. The expressions “preferably” and “preferentially” are used synonymously. The expressions “especially” and “especially preferably” are likewise used synonymously. Where documents are cited for the purposes of the present description, the entire content of these is intended to be part of the disclosure of the present invention. In the case of compositions, the percentage figures, unless stated otherwise, are based on the overall composition. Where figures are given in percent hereinafter, these are percentages by weight (% by weight) unless stated otherwise. Where average values are reported hereinafter, these are the numerical average, unless stated otherwise. Where measurements or physical properties, for example surface tensions or the like, are reported hereinafter, unless stated otherwise, these are measurements or physical properties measured at 25° C. and preferably at a pressure of 101 325 Pa (standard pressure) and preferably a relative air humidity of 50%. Where numerical ranges in the form of “from X to Y” are reported hereinafter, where X and Y are the limits of the numerical range, this is equivalent to the statement “from at least X up to and including Y”, unless stated otherwise. Statements of ranges thus include the range limits X and Y, unless stated otherwise.

Wherever molecules/molecule fragments have one or more stereocentres or can be differentiated into isomers on account of symmetries or can be differentiated into isomers on account of other effects e.g. restricted rotation, all possible isomers are included by the present invention.

The term “unsaturated” describes the presence of one or more carbon-carbon triple bonds and/or carbon-carbon double bonds that are not part of an aromatic ring.

Specific executions are defined hereinafter, and so features such as indices or structural constituents can be subject to restrictions by virtue of the execution. For all features unaffected by the restriction, the remaining definitions each remain valid.

The word fragment “poly” encompasses in the context of this invention not just compounds having at least 2 repeating units of one or more monomers in the molecule, but preferably also compositions of compounds having a molecular weight distribution and having an average molecular weight of at least 200 g/mol. This definition takes account of the fact that it is customary in the field of industry in question to refer to such compounds as polymers even if they do not appear to conform to a polymer definition as per OECD or REACH guidelines.

The various fragments in the formulae (I), (Ia), (Ib) (II), (III), (IV), (IVa), (IVb), (V) and (VI) below may be in a statistical distribution. Statistical distributions may have a blockwise structure with any number of blocks and any sequence or they may be subject to a randomized distribution; they may also have an alternating structure or else form a gradient along the chain, if there is one; in particular, they can also form any mixed forms in which groups of different distributions may optionally follow one another. The propyleneoxy units in formulae (II) and (III) and formulae (V) and (VI) may be bonded differently to the adjacent groups or atoms. In formula (VI) and formula (III), [CH₂CH(CH₃)O] is in each case independently a propyleneoxy radical of the [CH₂CH(CH₃)O] form and/or of the [CH(CH)CH₂O] form, but preferably a propyleneoxy radical of the [CH₂CH(CH)O] form. The formulae (I), (Ia), (Ib) (II), (III), (IV), (IVa), (IVb), (V) and (VI) describe compounds that are constructed from repeat units, for example repeating fragments, blocks or monomer units, and may have a molar mass distribution. The frequency of the repeat units is reported by indices. The indices are the numerical average over all repeat units. The indices a, b, c, d, g, o, p and optionally h used in the formulae should be regarded as statistical averages (number averages). The indices a, b, c, d, g, o, p and optionally h that are used and also the value ranges for the specified indices, are thus understood to mean average values of the possible statistical distribution of the structures and/or mixtures thereof that are actually present. The siloxanes to be used in accordance with the invention are preferably in the form of equilibrated mixtures. Specific embodiments may lead to restrictions to the statistical distributions as a result of the embodiment. There is no change in the statistical distribution for all regions unaffected by the restriction. Statements relating to preferred embodiments of a siloxane selected from the group of the siloxanes of the formulae (I), (Ia), (Ib), (IV), (IVa), (IVb) are also applicable mutatis mutandis to the other siloxanes in this group.

The present invention firstly provides a composition comprising at least one active microbiological ingredient and at least one siloxane of the formula (I)

M¹ₐM²_{b}D¹_{c}D²_{d} Formula (I)
with:
   M¹=R¹₃SiO_{1/2};
   M²=R¹₂R²₁SiO_{1/2};
   D¹=R¹₂SiO_{2/2};
   D²=R¹R²SiO_{2/2};
   a=0 to 2, preferably 1 to 2, especially preferably 2;
   b=0 to 2, preferably 0 to 1, especially preferably 0;
   c=1.5 to 100, preferably 2 to 60, especially preferably 2.5 to 40;
   d=0.5 to 25, preferably 0.7 to 15, further preferably 1 to 12, especially preferably 1.5 to 11;
   with the proviso that:
   a+b=0 to 2, preferably an integer selected from 0 and 2;
   a+b+c+d≥4;
   R¹ is in each case independently a monovalent hydrocarbyl radical, preferably having 1 to 12 carbon atoms, further preferably having 1 to 6 carbon atoms, even further preferably a linear or branched, aliphatic or aromatic, optionally unsaturated, monovalent hydrocarbyl radical, even further preferably methyl, ethyl, propyl or phenyl, especially preferably methyl;
   R² is in each case independently a monovalent polyether radical preferably bonded via a Si—C bond, further preferably a polyether radical of formula (II)

R³[O[AO]_{g}R⁴]ₕ Formula (II)
where AO is in each case independently an alkyleneoxy radical selected from ethyleneoxy, propyleneoxy and/or butyleneoxy,
especially preferably a polyether radical of formula (III)

—R³[O[CH₂CH₂O]ₒ[CH₂CH(CH₃)O]ₚR⁴]ₕ Formula (III)
with:
   g=2 to 35, preferably 3 to 30, further preferably 4 to 25, even further preferably 5 to 20, especially preferably 6 to 15;
   h=1 to 3, preferably 1 to 2, especially preferably 1;
   o=1 to 25, preferably 2 to 20, especially preferably 3 to 15;
   p=0 to 20, preferably 1 to 10, especially preferably 2 to 8;
   with the proviso that:

o+p=g;
R³ is in each case independently a polyvalent, preferably di- or trivalent, hydrocarbyl radical having preferably 2 to 12 carbon atoms, further preferably having 3 to 10 carbon atoms; optionally additionally containing heteroatoms, preferably 1 to 4 heteroatoms, further preferably 1 to 4 oxygen atoms; further preferably, R³ is additionally linear or branched, aliphatic or aromatic, optionally unsaturated; even further preferably, R³ is selected from the group consisting of:

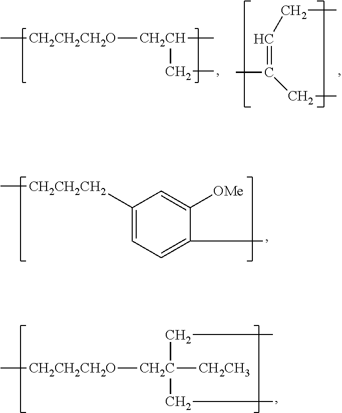

—CH₂CH(CH₃)CH₂—, —CH₂CH₂CH(CH₃)—,
—CH₂CH₂CH(CH₃)₂—, —CH₂CH₂CH₂—, —CH₂CH₂—;
even further preferably

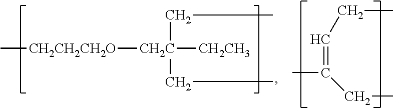

and —CH₂CH₂CH₂—:
especially preferably —CH₂CH₂CH₂—;
R⁴ is in each case independently hydrogen or a monovalent hydrocarbyl radical, preferably having 1 to 12 carbon atoms, further preferably a linear or branched alkyl radical, or —C(═O)R⁵, even further preferably hydrogen, methyl or acetyl, especially preferably hydrogen,
where:
   R⁵ is in each case independently hydrogen or a monovalent hydrocarbyl radical, preferably having 1 to 18 carbon atoms, further preferably linear or branched, aliphatic or aromatic, optionally unsaturated, especially preferably methyl and/or phenyl.

The composition may comprise siloxanes of formula (I) in which the sum total of the indices a and b is 2, i.e. a+b=2, and/or siloxanes of formula (I) in which the sum total of the indices a and b is 0, i.e. a+b=0. The siloxanes of formula (I) with a+b=2 are linear siloxanes. The siloxanes of formula (I) with a+b=0 are cyclic siloxanes.

A preferred embodiment of the present invention is a composition comprising at least one active microbiological ingredient and at least one linear siloxane of the formula (Ia)

M¹M²D¹_{c}D²_{d} Formula (Ia)

where M¹, M², D¹ and D² and the indices c and d are as defined for formula (I).

A further preferred embodiment of the present invention is a composition comprising at least one active microbiological ingredient and at least one cyclic siloxane of the formula (Ib)

D¹_{c}D²_{d} Formula (Ib)

where D¹ and D² and the indices c and d are as defined for formula (I).

A further preferred embodiment of the present invention is a composition comprising at least one active microbiological ingredient and at least one linear siloxane of the formula (Ia) and one cyclic siloxane of the formula (Ib).

Statements relating to preferred embodiments of the siloxanes of the formula (I) are also correspondingly applicable to siloxanes of the formulae (Ia) and (Ib).

It is further preferable that, for the at least one siloxane of formula (I), the sum total of the indices c and d, i.e. c+d, is from 2 to 125, preferably from 2.5 to 100, further preferably from 3.0 to 80, even further preferably from 3.5 to 60, even more preferably from 4 to 40, even more preferably from 4 to 10, especially from 4 to 6.

It is further preferable that, for the ratio of index c to d, i.e. for the quotient c/d of the indices c and d, of the at least one siloxane of formula (I), 1≤c/d≤6, further preferably 1≤c/d≤5, especially preferably 1≤c/d≤4.5.

It is further preferable that the following relation is applicable: c>d, further preferably c≥(d+1), especially preferably c≥(d+2).

Preferably, the composition comprises siloxanes of formula (I) in which R¹ is selected from methyl, ethyl, propyl or phenyl, especially preferably methyl, also in which R² is a polyether radical of formula (II), also in which R³ is the —CH₂CH₂CH₂— radical, and additionally in which R⁴ is hydrogen.

It is further preferable that the polyether radical of formula (III) has the ethyleneoxy and propyleneoxy units in particular ratios. It is therefore preferable that the ratio of index o to p, i.e. the quotient o/p of the indices o and p, is from 0.2 to 3.6, preferably from 0.6 to 3.2, further preferably from 1.0 to 2.8, even further preferably from 1.4 to 2.4, especially preferably from 1.9 to 2.8.

A further advantage of the compositions according to the invention is the biodegradability of the siloxanes of formula (I).

It is therefore further preferable that the mixture of the cyclic siloxanes of formula (I) has a biodegradability of not less than 60%, preferably of not less than 63% and especially preferably of not less than 65%, the maximum value being 100%.

Preferably, the compositions according to the invention do not include any non-biodegradable siloxanes.

Biodegradability is preferably determined by the OECD 301 F method. More preferably, biodegradability is determined in accordance with OECD 301 F after 28 days at 22° C. Further preferably, biodegradability is determined as in EP 3106033 A1, especially as described in the examples therein.

It is further preferable that the siloxanes of formula (I) and/or the compositions according to the invention have superspreading properties in water.

Spreading is examined here by applying a 50 μl droplet of the sample to be examined to a horizontally aligned standard polypropylene film (type: Forco-OPPB, from Van Leer, biaxially oriented polypropylene film). The droplet is applied with a micropipette. The maximum area of spread (spread area) and the time taken for the maximum extent of spread to be achieved (spreading time) were determined after the application. The spreading diameter is the diameter of the approximately circular area.

Preferably, the at least one siloxane of formula (I) and/or the composition according to the invention in water with water in excess in a mass ratio of 1:999 have a spread area of 25 to 70 cm², preferably of 30 to 60 cm², more preferably of 35 to 50 cm. Even more preferably, the at least one siloxane of formula (I) and/or the compositions according to the invention have the aforementioned spread area determined at a temperature of 25° C., a pressure of 1013.25 mbar and 50% relative air humidity.

Preferably, the at least one siloxane of formula (I) and/or the compositions according to the invention in water with water in excess in a mass ratio of 1:999 have a spreading diameter of 5 to 10 cm, preferably 6 to 9 cm, more preferably 7 to 8 cm. More preferably, the at least one siloxane of formula (I) and/or the compositions according to the invention have the aforementioned spreading diameter determined at a temperature of 25° C., a pressure of 1013.25 mbar and 50% relative air humidity.

Preferably, the at least one siloxane of formula (I) and/or the composition according to the invention in water with water in excess in a mass ratio of 1:999 have a spreading time of 90 to 280 s, preferably 100 to 260 s, further preferably 120 to 240 s, even further preferably 140 to 220 s, especially preferably 160 to 200 s. Even more preferably, the at least one siloxane of formula (I) and/or the compositions according to the invention have the aforementioned spreading time determined at a temperature of 25° C., a pressure of 1013.25 mbar and 50% relative air humidity.

It is further preferable that the composition is in liquid form, for example in the form of an oil dispersion (OD), dispersion concentrate (DC) or suspension concentrate (SC). This has the advantage that the composition is easy to handle. But it is also possible that the composition is solid, for example in the form of a wettable powder (WP) or of water-dispersible granules (WG).

The composition according to the invention comprises at least one active microbiological ingredient.

Preferably, the active microbiological ingredient is selected from the group consisting of microorganisms, organs of microorganisms and mixtures thereof. It is especially preferable that the microorganism is living and/or active.

It is further preferable that the active microbiological ingredient has a preferably antagonistic and/or hyperparasitic effect directed against a particular pathogen, preferably plant pathogen.

It is further preferable that the active microbiological ingredient increases resistance and/or stress tolerance and/or nutrient availability in plants.

The microorganisms in the context of the present disclosure include bacteria, fungi, algae, protozoa and viruses.

The microorganisms are accordingly selected from the group consisting of bacteria, fungi, algae, protozoa and viruses and mixtures thereof.

The microorganism is preferably selected from the group consisting of fungi and bacteria.

In a preferred embodiment, the microorganism is not selected from the group of viruses, especially not from the group consisting of viruses, algae and protozoa.

In a preferred embodiment, the active microbiological ingredient is selected from the group consisting of fungi, fungal organs, bacteria, bacterial organs and mixtures thereof.

The terms “active microbiological ingredient” and “active microbial ingredient” are used synonymously in the context of the present disclosure.

In a preferred embodiment, the active microbiological ingredient is selected from the group consisting of fungi, fungal organs and mixtures thereof.

It is further preferable that the fungal organs are selected from the group consisting of spores, conidia, blastospores, chlamydospores, sclerotia, hyphal segments and mixtures thereof.

Further preferably, the active microbiological ingredient is selected from the group consisting of the fungi Ampelomyces quisqualis, Aureobasidium pullulans, Beauveria bassiana, Beauvena brongniartii, Candida oleophila, Clonostachys rosea, Coniothyrium minitans, Gliocladium catenulatum, Gliocladium virens, Isaria fumosorosea, Isaria spp., Laetisana aivalis, Lecanicillium lecanii, Lecanicillium muscarium, Metarhizium anisopliae, Myrothecium verucana, Nomuraea rleyi, Paecilomyces lilacinus, Phlebiopsis gigantea, Phoma macrostoma, Purpureocillium lilacinus, Pythium oligandrum, Talaromyces flavus, Teratospema oligociadum, Trichoderma asperellum, Trichoderma atrovwride, Trichoderma gamsii, Trichoderma hamatum, Trichoderma harzianum, Trichoderma koningii, Tchoderma reesei, Trichoderma spp., Verticillium biguttatum, their fungal organs, and mixtures of these fungi and/or fungal organs.

More preferably, the active microbiological ingredient is selected from the group consisting of the fungi Ampelomyces quisqualis, Aureobasidium pullulans, Beauveria bassiana, Candida oleophila, Clonostachys rosea, Coniothyrium minitans, Gliocladium virens, Isaria fumosorosea, Lecanicillum muscarium, Metarhizium anisopliae, Myrothecium verrucaria, Purpureocillium lilacinus, Phlebiopsis gigantea, Trichoderma asperellum, Trichoderma atroviride, Trichoderma gamsii, Trichoderma hamatum, Trichoderma harzianum, Trichoderma koningii, Trichoderma reesei, their fungal organs, and mixtures of these fungi and/or fungal organs.

Particular preference is given to the use of the following fungi having antagonistic and/or hyperparasitic action against particular plant pathogens: Ampelomyces quisqualis, Beauveria bassiana, Beauveria brongniartii, Clonostachys rosea, Coniothyrium minitans, Gliocladium catenulatum, Isaria spp., Laetisaria arvalis, Lecanicillium lecanii, Lecanicillium muscarium, Metarhizium anisopliae, Nomuraea rileyi, Paecilomyces lilacinus, Phoma macrostoma, Pythium oligandrum, Talaromyces flavus, Teratosperma oligociadum, Trichoderma spp. and Verticillium biguttafum.

Fungi used with preference that improve nutrient availability in the soil or increase the resistance of the plants to stress factors (including pathogens and pests) are: Penicillium bilai, Trichoderma spp. and all species that can be classified in the group of the Mycorrhiza fungi.

Compositions in which the active microbiological ingredient is selected from the group consisting of fungi, fungal organs and mixtures thereof are particularly suitable for use as plant protection product, for use as biostimulant and/or for treatment of seed.

In a further preferred embodiment of the composition, the active microbiological ingredient is a bacterium or a mixture of various bacteria.

In a preferred embodiment of the composition according to the invention, the bacterium or the mixture of various bacteria is selected from the group consisting of Azospirillum brasilense, Azotobacter chroococum, Bacillus amyloliquefaciens, Bacillus firmus, Bacillus licheniformis, Bacillus mycoides, Bacillus pumilus, Bacillus subtilis. Bacillus thuringiensis, Bradyrhizobium spp., Burkholderia spp., Chromobacterium subtsugae, Gluconacetobacter spp., Pseudomonas chororaphis, Pseudomonas fluorescens, Pseudomonas syingae, Rhizobium spp., Streptomyces griseoviridis, Streptomyces lydicus and mixtures thereof. These compositions are particularly suitable for use as plant protection products, for use as biostimulant and/or for treatment of seed.

In a further preferred embodiment of the composition according to the invention, the bacterium or the mixture of various bacteria is selected from the group consisting of Lactobacillus gasseri, Lactobacillus paracasei, Lactobacillus plantarum, Lactobacillus crispatus, Lactobacillus casei, Lactobacillus animalis, Lactobacillus rhamnosus, Lactobacillus pentosus, Lactobacillus reuteri, Lactococcus lactis, Bacillus pumilus, Bacillus lichenifonis, Bacillus coagulans, Bacillus cereus, Bacillus subtilis, Bacillus amyoliquefaciens, Clostridium butyricum, Enterococcus faecium, Streptococcus faecium, Lactobacillus acidophilus, Lactobacillus salivarus, Lactobacillus fermentum, Lactobacillus johnsonii, Lactobacillus helveticus, Streptococcus thermophiles, Pediococcus acidilactici, Bifidobacterium lactis, Bifidobacterium adolescentis, Bifidobacterium lactobacillus, Bifidobacterium animalis, Bifidobacterium longum, Bifidobacterium infantis and mixtures thereof. These compositions are particularly suitable for use as probiotic in foods and/or animal feeds.

In a further preferred embodiment of the composition, the active microbiological ingredient is selected from the group consisting of lactobacilli, bifidobacteria, Enterococcus faecalis, Enterococcus faecium and the yeast fungi Saccharomyces boulardii and Saccharomyces cerevisiae and mixtures thereof. These compositions may be suitable, for example, for use as probiotic medicament. For some disorders and fields of use, the efficacy of probiotic medicaments is comparatively well-researched. These include various chronic inflammatory bowel disorders, various diarrhoeal disorders, chronic constipation, prevention of allergies and infections in prematuer babies, prevention of neurodermatitis, infections of the throat, nose, ears, urinary tract infections and dental caries.

In a further preferred embodiment of the composition, the active microbiological ingredient is a virus or a mixture of different viruses, preferably selected from the group of the baculoviruses, further preferably from the nucleopolyhedrovirus and granulovirus genera. In a preferred embodiment of the composition, the active microbiological ingredient selected is the CpGV virus (Cydia pomonella granuovirus). This virus is used, for example, for protection from codling moth caterpillars in pomiculture. In a further preferred embodiment of the composition, the active microbiological ingredient selected is the HearNPV virus (Helicoverpa armigera nucleopolyhedrovirus). This virus acts specifically against the larvae of the cotton bollworm and is used, for example, for production of cotton plants.

Also preferred in accordance with the invention is a composition in which the active microbiological ingredient is a mixture of the abovementioned microorganisms and/or organs thereof.

Preference is given to a composition in which the active microbiological ingredient comprises spores, preferably fungal spores and/or bacterial spores, especially spores of Trichoderma harzianum and/or of Bacillus amyloliquefaciens.

Preference is also given to a composition in which the active microbiological ingredient comprises vegetative cells, especially vegetative cells of Pseudomonas fluorescens.

Preferably, the proportion by mass of water based on the total mass of the composition is less than 10%, preferably less than 5%, especially preferably less than 1%. This increases the viability and/or germinability of the active microbial ingredient present. In addition, the hydrolysis of the siloxanes is thus reduced.

It is further preferable that the proportion by mass of active microbiological ingredients based on the total mass of the composition is less than 50%, preferably from 5% to 40%, further preferably from 10% to 30%, especially preferably from 15% to 25%.

It is further preferable that the proportion by mass of siloxanes of formula (I) based on the total mass of the composition is more than 50%, preferably from 60% to 95%, further preferably from 70% to 90%, especially preferably from 75% to 85%.

It is further preferable that the ratio of the mass of siloxanes of formula (I) to the mass of active microbiological ingredients is from 1:1 to 20:1, preferably from 2:1 to 10:1, further preferably from 3:1 to 7:1, especially preferably from 4:1 to 6:1, for example 5:1.

A further advantage is the improved storability of the compositions according to the invention.

It is therefore further preferable that the proportion of germinable spores after storage, determined as described in the examples, after 7 days is at least 50%, further preferably at least 60%, even more preferably at least 70%, even further preferably at least 80%, especially at least 90%, based on the starting value.

It is further preferable that the proportion of germinable spores after storage, determined as described in the examples, after 14 days is at least 40%, further preferably at least 50%, even more preferably at least 60%, even further preferably at least 70%, especially at least 80%, based on the starting value.

It is further preferable that the proportion of germinable spores after storage, determined as described in the examples, after 21 days is at least 20%, further preferably at least 30%, even more preferably at least 40%, even further preferably at least 50%, especially at least 60%, based on the starting value.

It is therefore further preferable that the proportion of germinable spores after storage, determined as described in the examples, after 28 days is at least 5%, further preferably at least 10%, even more preferably at least 15%, especially at least 20%, based on the starting value.

A further advantage is the improved germination rate of the compositions according to the invention.

It is therefore further preferable that the germination rate, determined as described in the examples, is at least 10%, further preferably at least 20%, even further preferably at least 30%, even further preferably at least 40%, even further preferably at least 50%, even further preferably at least 60%, even further preferably at least 70%, even further preferably at least 80%, especially at least 90%, based on the starting value.

The invention further provides a process for producing the composition according to the invention, characterized in that at least one active microbiological ingredient, preferably selected from the group consisting of microorganisms, organs of microorganisms and mixtures thereof, and at least one siloxane of formula (I) are mixed.

It is preferable that the at least one active microbiological ingredient, preferably selected from the group consisting of microorganisms, organs of microorganisms and mixtures thereof, is suspended and/or dispersed in at least one siloxane of formula (I).

It is further preferable that the microorganism(s) is/are cultivated and the cultivated microorganism(s) is/are suspended and/or dispersed in at least one siloxane of formula (I).

The microorganism is preferably cultivated on a nutrient medium suitable for the purpose by methods known per se, for example submerged fermentation or solid fermentation. Preferably, the cultivated microorganism is processed by suitable separation, drying, grinding and/or dispersion methods. Preferably, after the cultivation, the microorganism and/or its organs that are used with preference is/are preferably separated from the culture substrate. In a particularly preferred variant, the culture substrate over which the microorganism has grown (especially in the case of use of solid culture substrate) is dried beforehand. In another variant, the microorganism or its organs used with preference, after they have been separated from the culture substrate, can be dried, for example, with the aid of freeze-drying or spray-drying methods. After the separation and any drying, the microorganism and/or its organs is/are suspended and/or dispersed in a siloxane of formula (I).

It is further preferable that the microorganism, preferably selected from the group of the fungi, is processed by grinding and/or dispersing methods. In this case, the cultivation is followed, prior to the separation of the microorganism and/or its organs that are used with preference, by processing of the culture substrate on which they have grown by a suitable dispersion method or, after drying, by a suitable grinding method.

Preferably, there is then a subsequent separation of the microorganism or of its organs that are used with preference by methods known per se, such as sieving, filtration, windsifting, decanting or centrifugation methods. A preferred embodiment of the production process is accordingly characterized in that the at least one microorganism and/or the at least one organ of the microorganism, preferably selected from the group consisting of fungi and fungal organs, after processing, is isolated by sieving, filtration, windsifting and/or centrifugation methods.

Preferably, the composition is produced by mixing the at least one microorganism and/or its organs into the at least one siloxane of formula (I), preferably in a mixing tank using a stirrer. This preferably affords a liquid composition, for example an oil dispersion (OD), suspension concentrate (SC) or dispersion concentrate (DC). By selection of suitable siloxanes and/or use of appropriate viscosity regulators, it is possible to adjust the viscosity such that at least unity reduced separation, if any, of the microorganisms that have been mixed into the liquid formulation, preferably an SC and DC formulation, is observed.

It is further preferable that the process for producing a composition according to the invention comprises at least one process step in which a composition comprising at least one SiH-functional siloxane of the formula (IV)

M¹ₐM³_{b}D¹_{c}D³_{d} Formula (IV)
with:
   M¹=R¹₃SiO_{1/2};
   M³=R¹₂HSiO_{1/2};
   D¹=R¹₂SiO_{2/2};
   D³=R¹HSiO_{2/2};
where:
   the indices a, b, c and d are as defined for formula (I);
   R¹ is as defined for formula (I);
is reacted in the manner of a hydrosilylation with at least one unsaturated, preferably terminally unsaturated, polyether, further preferably with a polyether of formula (V)

R⁶[O[AO]_{g}R⁴]ₕ Formula (V);
where AO is in each case independently an alkyleneoxy radical selected from ethyleneoxy, propyleneoxy and/or butyleneoxy,
even further preferably with a polyether of formula (VI)

R⁶[O[CH₂CH₂O]ₒ[CH₂CH(CH₃)O]R⁴]ₕ Formula (VI)
where:
   the indices g, h, o, p are as defined for formulae (II) and (III);
   R⁴ is as defined for formulae (II) and (III);
   R⁶ is in each case independently a mono- or polyvalent, preferably a mono- or divalent, unsaturated, preferably terminally unsaturated, hydrocarbyl radical, preferably having 2 to 12 carbon atoms, further preferably having 3 to 10 carbon atoms; optionally additionally containing heteroatoms, preferably 1 to 4 heteroatoms, preferably 1 to 4 oxygen atoms; further preferably, R⁶ is additionally linear or branched, aliphatic or aromatic, but in any case unsaturated; even further preferably, R⁶ is selected from the group consisting of:

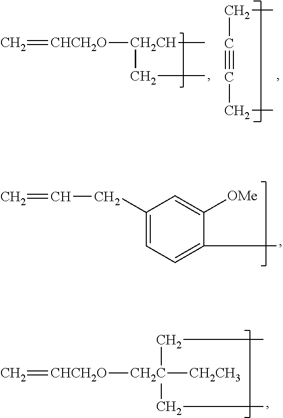

CH₂═C(CH₃)CH₂—, CH₂═CHCH(CH₃)—,
CH₂═CHCH(CH₃)₂—, CH₂═CHCH₂—, CH₂═CH—;
even further preferably

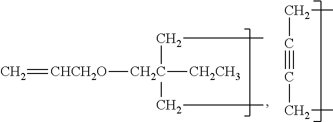

and CH₂═CHCH₂—;
especially preferably CH₂═CHCH₂—:
so as to obtain a composition comprising the at least one siloxane of formula (I).

This composition or this at least one siloxane of formula (I) is then used in the process according to the invention, i.e. for preparation of the composition according to the invention.

The composition may comprise siloxanes of formula (IV) in which the sum total of the indices a and b is 2, i.e. a+b=2, and/or siloxanes of formula (IV) in which the sum total of the indices a and b is 0, i.e. a+b=0. The siloxanes of formula (IV) with a+b=2 are linear siloxanes. The siloxanes of formula (IV) with a+b=0 are cyclic siloxanes.

It is further preferable that the process for producing a composition according to the invention comprises at least one process step in which a composition comprising at least one linear SiH-functional siloxane of the formula (IVa)

M¹M³D¹_{c}D³_{d} Formula (IVa)

where M¹, M³, D¹ and D³ and the indices c and d are as defined for formula (IV) is reacted in the manner of a hydrosilylation with at least one unsaturated, preferably terminally unsaturated, polyether, as defined above, so as to obtain a composition comprising at least one linear siloxane of formula (Ia).

It is further preferable that the process for producing a composition according to the invention comprises at least one process step in which a composition comprising at least one cyclic SiH-functional siloxane of the formula (IVb)

D¹_{c}D³_{d} Formula (Vb)

where D¹ and D³ and the indices c and d are as defined for formula (IV) is reacted in the manner of a hydrosilylation with at least one unsaturated, preferably terminally unsaturated, polyether, as defined above, so as to obtain a composition comprising at least one cyclic siloxane of formula (Ib).

It is further preferable that the process for producing a composition according to the invention comprises at least one process step in which a composition comprising at least one linear SiH-functional siloxane of the formula (IVa) and at least one cyclic SiH-functional siloxane of the formula (IVb) is reacted in the manner of a hydrosilylation with at least one unsaturated, preferably terminally unsaturated, polyether, as defined above, so as to obtain a composition comprising at least one linear siloxane of the formula (Ia) and at least one cyclic siloxane of formula (Ib).

Statements relating to preferred embodiments of the siloxanes of the formula (IV) are also correspondingly applicable to siloxanes of the formulae (IVa) and (IVb).

It is further preferable that, for the at least one siloxane of formula (I), the sum total of the indices c and d, i.e. c+d, is from 2 to 125, preferably from 2.5 to 100, further preferably from 3.0 to 80, especially preferably from 3.5 to 60, especially from 4 to 40.

It is further preferable that, for the ratio of index c to d, i.e. for the quotient c/d of the indices c and d, of the at least one siloxane of formula (I), 1≤c/d≤6, further preferably 1≤c/d≤5, especially preferably 1 s c/d≤4.5.

It is further preferable that the following relation is applicable: c>d, further preferably c≥(d+1), especially preferably c≥(d+2).

Preferably, the SiH-functional siloxanes of formula (IV) that are used in the process according to the invention have an R¹ radical selected from methyl, ethyl, propyl or phenyl, especially preferably methyl, and the polyether of formula (V) used in the process according to the invention has CH₂═CHCH₂— as R⁶ radical and hydrogen as R⁴ radical.

It is further preferable that the polyether of formula (V) has the ethyleneoxy and propyleneoxy units in particular ratios. It is therefore preferable that the ratio of index o to p, i.e. the quotient o/p of the indices o and p, is from 0.2 to 3.6, preferably from 0.6 to 3.2, further preferably from 1.0 to 2.8, even further preferably from 1.4 to 2.4, especially preferably from 1.9 to 2.8.

The polyethersiloxanes of formula (I) are prepared by means of hydrosiylation in the manner known to the person skilled in the art. This involves reacting the correspondingly SiH-functional siloxanes of formula (IV) with unsaturated polyethers by known methods.

The hydrosilylation reaction in the process according to the invention is preferably catalysed with the aid of the platinum group catalysts familiar to those skilled in the art, more preferably with the aid of Karstedt catalysts.

The hydrosilylation reaction in the process according to the invention is preferably brought to a full conversion in relation to the hydrogen content of the SiH-functional siloxane of the formula (IV). In the context of the present disclosure, full conversion is understood to mean that the conversion of SiH functions is >99%. Detection is effected in a manner familiar to the person skilled in the art, preferably by gas-volumetric means after alkaline breakdown. This can be done, for example, by reacting a sample of the reaction mixture with a butanolic sodium butoxide solution (sodium butoxide content: 5% by weight) and concluding the amount of SiH functions still present from the amount of hydrogen formed.

Optionally, the at least one SiH-functional siloxane of formula (IV), prior to the hydrosilylation, is purified in that it is subjected to a suitable thermal separation process.

Likewise optionally, the process product according to the invention is purified, preferably by means of a thermal separation process.

Thermal separation processes are known by this term to those skilled in the art and include all processes based on the establishment of a thermodynamic phase equilibrium. Preferred thermal separation processes are selected from the list comprising distillation, rectification, adsorption, crystallization, extraction, absorption, drying and freezing-out, particular preference being given to methods of distillation and rectification.

The inventive compositions can be produced by the prior art methods, but preferably by the process according to the invention.

The synthesis of polyethersiloxanes is familiar to the person skilled in the art. The synthesis of cyclic polethersiloxanes is described, for example, in DE 19631227. This describes both the synthesis of isolated cyclic polyethersiloxanes proceeding from isolated cyclic SiH-functional siloxanes and the synthesis of mixtures of cyclic polyethersiloxanes proceeding from mixtures of cyclic SiH-functional siloxanes. The synthesis of linear polyethersiloxanes is disclosed, for example, in US 20120245305, WO 2013066983 or U.S. Pat. No. 6,987,157.

The SiH-functional siloxanes can likewise be obtained by known methods via equilibration/cyclization and optionally distillation. The preparation of linear SiH-functional siloxanes by means of equilibration with trifluoromethanesulfonic acid is described, for example, in U.S. Pat. No. 5,578,692. The preparation of cyclic SiH-functional siloxanes is described, for example, in U.S. Pat. Nos. 3,714,213, 4,895,967 and 5,247,116. In this way, it is possible to prepare mixtures of SiH-functional siloxanes. By means of the distillation, it is possible to separate the cyclic SiH-functional siloxanes from one another if required, i.e. to isolate them, and to use them further in the form of a single compound.

It is in turn possible to use the SiH-functional siloxanes separated by fractional distillation, for example, to prepare mixtures of SiH-functional siloxanes. These mixtures can be used to prepare the siloxanes of formula (I) according to the invention by means of hydrosilylation reaction.

But the siloxanes of formula (I) can also be prepared from their individual compounds, i.e. from individual isolated siloxanes of formula (I). This can be accomplished, for example, by mixing siloxanes of formula (I). The siloxanes of formula (I) can be prepared from the corresponding SiH-functional siloxanes, isolated by fractional distillation for example, via a hydrosilylation.

It is advantageous when SiH-functional siloxanes are used in the form of a mixture and it is possible to dispense with a complex fractional distillation.

The present invention further provides for the use of the composition according to the invention and/or of the process products according to the invention for the treatment of plants, of seed and/or of soils, and/or for use as biostimulant.

Preferably, the composition according to the invention and/or the process product according to the invention is used as biological plant protection product, biological plant fortification product or biological soil improvement product; more preferably, the composition according to the invention and/or the process product according to the invention is used for plant protection.

In the case of use for plant protection, for the treatment of seed and/or as a biostimulant, the composition is preferably mixed or watered into the soil or applied to the plant or to the seed. According to the intended end use, the composition here is optionally diluted with water to the use concentration.

Preferably, the compositions according to the invention and/or the process products according to the invention are used as formulation, preferably as plant protection formulation, for spray liquors. Preferably, the proportion by mass here of all siloxanes of formula (I) based on the total mass of the spray liquor is from 0.001% to 1%, further preferably from 0.01% to 0.5%.

Preferred use concentrations here are between 0.001% and 1% by volume, preferably between 0.01% and 0.5% by volume and more preferably between 0.02% and 0.15% by volume (also corresponding to about 0.1% by weight) of the spray liquor.

Preferably, the spray liquor is applied to the plant via an irrigation system selected from the group consisting of micro-irrigation systems, sprinkler systems and drip systems.

For their use on plants or plant parts, plant protection formulations are, in most cases, diluted with water before the usual spraying through nozzles, and contain, besides the active component, other adjuvants too, such as emulsifiers, dispersing aids, antifreeze agents, antifoams, biocides and surface-active substances such as surfactants. Active substances, especially fungicides, insecticides and nutrients, alone or in combination and having been provided with the other auxiliaries specified above, can also be applied by various methods to seeds (seed) of plants. Such methods are also referred to as seed treatment methods. The treatment of seed with fungicides and insecticides can protect plants in the early stage of growth from diseases and attack by insects.

The plant protection formulations can also be applied to the plants by means of insects that pollinate plants, called “pollinators”, for example hornets or bees. The composition here is optionally diluted with water to the use concentration. But preference is given to using the composition undiluted. The spreading of chemical plant protection products by means of pollinating insects is described, for example, in WO 2011026983 A1. Biological plant protection products can also be spread in a corresponding manner. It is advantageous here when the pollinators are not impaired or damaged by the active microbiological ingredient or the composition.

If biocides are employed in the formulations, they are selected such that they are not harmful to the microorganisms of the compositions according to the invention. This means that the microorganisms in the formulation are restricted only to a minor degree, if at all, in their viability and/or germinability. Preferably, viability and/or germinability is maintained to an extent of at least 80%, preferably at least 90%, more preferably to an extent of at least 95%, 2 hours after the formulation has been made up. The study is effected according to AOAC® Official Method 2014.05 as described in the examples. The maximum value is determined by plating out the formulation immediately after it has been made up and counting the colony-forming units (CFU), and corresponds to 100%.

A composition containing conidia of Paecilomyces lilacinus as active microbial ingredient can be used for the biological control of phytoparasitic nematodes. When spores of Talaromyces flavus are used, the preparation can be used for control of Verticillium dahliae, a pathogen that causes economically relevant wilting in cotton. Compositions containing spores of Nomuraea rileyi can be used to control the caterpillars of various damaging butterfly species, for example Helicoverpa armigera and Spodoptera exigua. The employment of the composition using conidia of Penicillum bilaii increases the availability of mineral phosphorus in the soil.

Preferred agricultural fields of use of the composition according to the invention and/or of the process products according to the invention are arable farming, horticulture and cultivation of ornamentals, viticulture and cotton growing. Particular preference is given to fruit and vegetable growing. Preferred fruit is pome fruit, stone fruit, berry fruit and shelled fruit. Preferred vegetables are root vegetables, shoot vegetables, tuber vegetables, onion-type vegetables, leafstalk vegetables, leaf vegetables, leaf lettuces, seed vegetables and fruit vegetables.

In the case of use of the composition
i) for the treatment of plants; or
ii) for the treatment of seed; or
iii) for the treatment of soils; or
iv) as biostimulant;
   the composition is preferably used as a formulation for spray liquors, where the proportion by mass of all siloxanes of formula (I) based on the total mass of the spray liquor is 0.001% to 1%.

The present invention further provides for the use of the composition according to the invention and/or of the process products according to the invention as probiotic in foods and/or animal feeds. Probiotic foods and/or animal feeds typically contain bacteria and of fungi as active microbial ingredient. The probiotic foods include, for example, yoghurt preparations, kefir preparations, soured milk preparations and vegetables fermented in soured milk. The active microbial ingredient here displays a health-promoting effect in the intestine.

The present invention further provides for the use of the composition according to the invention and/or of the process products according to the invention as probiotic food supplement and/or probiotic animal feed additive.

The present invention further provides a composition according to the invention and/or a process product according to the invention for use as probiotic medicament.

The compositions and/or process products according to the invention have numerous advantages over the prior art.

The compositions and/or process products according to the invention show a higher survival rate of the microorganisms, also compared to compositions based on the known polyether-modified trisiloxanes.

The increased storability of the compositions according to the invention and/or of the process products according to the invention leads especially to an increase in biological activity by comparison, i.e. an increase in the effect of the composition.

A further advantage is that the compositions according to the invention and/or the process products according to the invention can be stored at room temperature over many weeks. This simplifies transport and storage. The composition is stored and transported, preferably with exclusion of air, in bottles, pouches, canisters or drums that have been sealed airtight.

Especially in the case of dispersion concentrates or suspension concentrates that contain fungal spores and liquid siloxanes of formula (I), viability and/or germinability can be distinctly improved over the prior art.

The invention is also advantageous since the composition, especially in the form of a suspension concentrates or dispersion concentrate, can preferably be incorporated readily into water. Preferably, the siloxane of formula (I) is fully soluble in water at room temperature, and so the active microbial ingredient is suspended/dispersed in the aqueous solution of the siloxane of formula (I). The product according to the invention preferably lowers the surface tension of water to <30 mN/m (surface tension by Wilhelmy plate method, determined with a Kruss K 12 tensiometer) and makes it possible to suspend the hydrophobic microorganisms in water. For this purpose, generally no further surfactants are needed. As a result of the low surface tension of the water, the spray droplets stick to hydrophobic leaf surfaces and also wet hydrophobic soils. This leads to better penetration and distribution of the microorganisms into the soil. Application is simple owing to the preferably good water solubility of the siloxanes of formula (I) and associated good suspendability/dispersibility of the active microbial ingredient, i.e. more particularly of the microorganism and/or its organs that are used with preference in the spray liquor. Application can be effected by means of spraying methods or by injection into the irrigation system, which assures homogeneous distribution of the effective agent in the soil, on the plant, on the seed and/or on the harmful organisms to be controlled.

A further improvement over the prior art is that the microorganisms and/or their organs remain viable and/or germinable for much longer in the ready-to-use aqueous dilutions than in the aqueous dilutions based on the prior art. In practice, formulations of microbiological plant protection products are diluted in water by the user prior to use. For this purpose, the plant protection products are typically added to a tank with water as ingredient and distributed in what is called the spray liquor while stirring. The adjuvants are typically employed in the spray liquor in concentrations of 0.001% by volume to 1% by volume. When trisiloxanes are used as carrier liquid for microorganisms as disclosed in EP2713718, this corresponds to a dilution of 1:1000; the concentration of trisiloxane in the spray liquor thus corresponds to an order of magnitude of 0.1% by volume. However, in EP2713718, the survival rate of the microorganisms was determined in a concentration of 0.01% by volume. After the application of the spray liquor, the effective or bioavailable concentration of adjuvant, as a result of evaporation and concentration, can even be considerably higher. Thus, a higher concentration of adjuvant will affect and be potentially harmful to the microorganisms the longer the sprayed droplet is subject to evaporation.

Prior to application, formulations of fungal spores can be made up in a preliminary mixture with water in order to accelerate germination and reduce infection time (cf. H. D. Burges: Formulation of Microbial Biopesticides, Springer, 1998). Some manufacturers of microbial products (e.g. Remedier® from Isagro, Naturalis® from CBC Europe, FZB24 from ABTEP GmbH) likewise recommend activating the spores in the formulation prior to spray application. For this purpose, the formulation is diluted in a relatively small amount of water in a bucket (by a factor of 3-50) and left to stand for 2 to 24 hours prior to spraying. Since the microorganisms are particularly sensitive in this phase, it is advisable to use siloxanes of formula (I) that are biocompatible in the formulation without any adverse effects on the microorganism.

The compositions according to the invention and/or process products according to the invention thus feature higher viability of microorganisms present at room temperature or slightly elevated temperatures. Thus, they are uncomplicated to store and to transport, and do not require any cooling in order to ensure that a sufficiently high concentration of germinable or viable microorganisms arrives at the target locus on the plant or in the soil. In the ready-to-use aqueous dilutions, the compositions according to the invention do not impair the germination or growth of microorganisms at the target locus.

The present invention is elucidated in detail by the working examples which follow without restricting the invention to the specific parameters described therein.

### EXAMPLES

### General Methods:

### Determination of the Composition of the SiH-Functional Siloxanes:

The siloxanes can be characterized with the aid of ¹H NMR and ²⁹Si NMR spectroscopy. These methods, especially taking account of the multiplicity of the couplings, are familiar to the person skilled in the art.

The proportion by mass of the SiH-functional siloxanes can be determined with the aid of a gas chromatography method (GC method) in which the substances are separated according to their boiling point and detected by means of a thermal conductivity detector. This is done by analysing an aliquot of the sample to be examined without further dilution by means of GC. This is conducted in a gas chromatograph equipped with a split/splitless injector, a capillary column and a thermal conductivity detector, under the following conditions:

| | |
|---|---|
| Injector: | 290° C., split 40 ml |
| Injection volume: | 1 μl |
| Column: | 5 m * 0.32 mm HP5 1 μm |
| Carrier gas: | helium, const, flow, 2 ml/min |
| Temperature program: | 1 minute at 80° C., then 80° C.-300° C. |
| | at 30° C./min, then conditioning |
| | at 300° C. for 10 minutes. |
| Detector: | TCD at 320° C. |
| | Make-up gas 6 ml/min |
| | Reference gas 18 ml/min |

The SiH-functional siloxanes are separated according to their boiling point. The proportion by mass of the individual substances is determined as the percentage of the peak areas determined for the respective substance by comparison with the total area of all substances detected (area % method).

### Determination of the Number of Colony-Forming Units (CFU)

To determine the number of colony-forming units (CFU), 1.0 g of the compositions according to the invention is diluted with sterile physiological saline (0.9% by weight of NaCl in water) in a decimal dilution series down to the level of 10⁻⁸. Typically, the 10⁻⁸, 10⁻⁷ and 10⁻⁸ dilution levels (1.0 ml of each) are plated onto ready-made nutrient medium (Compact Dry YM for yeasts and mould fungi or Compact Dry Total Count from Nissui Pharmaceutical Co., Ltd.). Fungal spores are incubated at 25° C. for three days, bacteria at 30° C. for one day. Plates on which 10-100 CFU are visible are evaluated.

### SiH-Functional Siloxanes:

### Example a-0

29.7 g of poly(methylhydrosiloxane) (CAS: 63148-57-2, Gelest Inc., Code HMS-992 M_{eq.}=63.8 g/mol SIH, i.e. 63.8 g based on the number of SiH groups) were mixed with 32.7 g of hexamethyldisiloxane and 37.6 g of octamethylcyclotetrasiloxane, and 0.1 g of trifluoromethanesulfonic acid (purity: 99%) was added. The mixture was stirred at room temperature for 24 h. Subsequently, 2 g of NaHCO₃ were added and the mixture was stirred for 4 h. The mixture was filtered. A clear liquid was obtained. The siloxane was characterized with the aid of ²⁹Si NMR spectroscopy. A siloxane of the general empirical formula Me₃SiO[SiMe₂O]_{2.4}[SiMeHO]_{2.2}SiMe₃ was obtained.

### Example b-0

800 g of octamethylcyclotetrasiloxane were mixed with 180 g of polymethylhydrosiloxane (CAS 63148-57-2. M_{eq.}=63.8 g/mol SiH, i.e. 63.8 g based on the number of SiH groups) and 35 g of hexamethyldisiloxane under a protective gas blanket. Subsequently, 2 g of trifluoromethanesulfonic acid (purity: 99%) were added. The mixture was stirred at room temperature for 24 h. Subsequently, 20 g of NaHCO₃ were added and the mixture was stirred for 4 h. Thereafter, the product was filtered and the filtrate obtained was freed of volatile constituents on a rotary evaporator at 130° C. at a pressure of 1 mbar. The product obtained was characterized with the aid of ²⁹Si NMR spectroscopy and the GC method. The empirical formula is ([SiMe₂O]_{0.61}[SiMeHO]_{0.19})ₓ (x=4 to 6). The distillate contains 92% by weight of SiH-functional cyclic siloxanes, i.e. siloxanes of formula (I) with a+b=0. The siloxanes are in the form of a mixture of 4-, 5- and 6-membered rings. The siloxanes are thus of formula (I) with c+d=4, 5 or 6.

### Example c-0

17.8 g of poly(methylhydrosiloxane) (CAS: 63148-57-2, Gelest Inc., Code HMS-992 M_{eq.}=63.8 g/mol SiH, i.e. 63.8 g based on the number of SiH groups) were mixed with 3.5 g of hexamethyldisiloxane and 78.7 g of octamethylcyclotetrasiloxane, and 0.1 g of trifluoromethanesulfonic acid (purity: 99%) was added. The mixture was stirred at room temperature for 24 h. Subsequently, 2 g of NaHCO₃ were added and the mixture was stirred for 4 h. The mixture was filtered. A clear liquid was obtained. The siloxane was characterized with the aid of ²⁹Si NMR spectroscopy. A siloxane of the empirical formula Me₃SiO[SiMe₂O]₃₈[SiMeHO]₁₀SiMe₃ was obtained.

### Polyethersiloxanes

### General Remarks:

The polyethersiloxanes were prepared in the examples which follow by hydrosilylation. This was done by reacting SiH-functional siloxanes with an unsaturated polyether. The hydrosilylation reaction was conducted in the presence of a complete platinum(0)-1,3-divinyl-1,1,3,3-tetramethydisiloxane solution in xylene (purchased from Sigma-Aldrich, Pt content: 2% by weight) as Karstedt catalyst. The hydrosilylation reaction was brought to full conversion in relation to the hydrogen content of the SiH-functional siloxanes. In the context of the present invention, full conversion is understood to mean that more than 99% of the SiH functions were converted. Detection is effected in the manner familiar to the person skilled in the art by gas-volumetric means after alkaline breakdown.

### Example a-1

152.3 g of a polyether of the empirical formula CH₂═CHCH₂O[C₂H₅O]₆[CH₂CH(CH₃)O]₃H were mixed with 47.7 g of an SiH-functional linear siloxane from Example a-0 in a 500 ml three-neck flask with precision glass stirrer and reflux condenser under a nitrogen blanket. The mixture was then heated to 90° C. Subsequently, 0.04 g of a solution of the Karstedt catalyst in xylene (Pt content 2% by weight) was added to the mixture. An exothermic reaction set in. The mixture was then stirred at 90° C. for 2 h. A clear liquid was obtained. It was no longer possible to detect any SiH functions by gas-volumetric means. The conversion of SiH functions was 100%. A siloxane of the empirical formula Me₃SiO[SiMe₂O]_{2.4}[SiMeR²O]_{2.2}SiMe₃ with R²═—CH₂CH₂CH₂O[C₂H₅O]₆[CH₂CH(CH₃)O]₃H was obtained.

### Example b-1

109.9 g of a polyether of the empirical formula CH₂═CHCH₂O[C₂H₅O]₆[CH₂CH(CH₃)O]₃H were mixed with 60 g of the cyclic SiH-functional siloxane of the empirical formula ([SiMe₂O]_{0.81}[SiMeHO]_{0.19})ₓ (x=4 to 6) from Example b-0 in a 250 ml three-neck flask with precision glass stirrer and reflux condenser under a nitrogen blanket. The mixture was then heated to 90° C. Subsequently, 0.03 g of a solution of the Karstedt catalyst in xylene (Pt content 2% by weight) was added to the mixture. An exothermic reaction set in. The mixture was then stirred at 90° C. for 2 h. A clear liquid was obtained. The conversion of SiH functions was 100%. A siloxane of the empirical formula ([SiMe₂O]_{0.81}[SiMeR²O]_{0.19})ₓ (x=4 to 6) with R²═—CH₂CH₂CH₂O[C₂H₅O][CH₂CH(CH₃)O]₃H was obtained.

### Example a-1

262 g of a polyether of the empirical formula CH₂CHCH₂O[C₂H₅O]_{13.9}[CH₂CH(CH₃)O]_{5.3}H were mixed with 70 g of a siloxane of the empirical formula Me₃Si[SiMe₂O]₃₈[SiMeHO]₁₀SiMe₃ from Example c-0 in a 500 ml three-neck flask with precision glass stirrer and reflux condenser under a nitrogen blanket. The mixture was heated to 90° C. Subsequently, 0.16 g of a solution of the Karstedt catalyst in xylene (Pt content 2% by weight) was added to the mixture. An exothermic reaction set in. The mixture was then stirred at 90° C. for 2 h. A clear liquid was obtained. The conversion of SiH functions was 100%. A siloxane of the empirical formula Me₃SiO[SiMe₂O]₃₈[SiMeR²O]₁₀SiMe₃ with R²═—CH₂CH₂CH₂O[C₂H₅O]_{13.9}[CH₂CH(CH₃)O]_{5.3}H was obtained.

Production of the Compositions with T. harzianum Spores

### Examples a-2 to c-2

Spores of the Trichoderma harzianum fungus were sourced from Rhizo-Mic UG and contained, according to elemental analysis, apart from the spores, about 75% by weight of SiO₂. The powder contained 3×10⁹ germinable spores/g of product. The compositions of polyethersiloxanes and spores of T. harzianum were produced as follows: 5.00 g of spores were weighed into a 50 ml centrifuge tube (e.g. sterile 50 ml tubes from Greiner Bio-One GmbH), and blanketed with 20.00 g of the polyethersiloxane a-1, b-1 or c-1. The mixture was mixed on a vortex shaker (lab dancer from ika) for 30 seconds. After homogenization with a spatula, the composition, after a wait time of 15 minutes, was mixed again on a vortex shaker for 30 seconds. The composition produced contained 6×10⁸ germinable spores/g.

### Determination of Storage Stability

The compositions a-2 to c-2 produced, which comprise spores of Trichoderma harzianum in polyethersiloxanes, were incubated at 40° C. for four weeks, and the number of colony-forming units was determined immediately after production (starting value) and after 7, 14, 21 and 28 days. The corresponding procedure was followed with the comparative examples. The number of colony-forming units (CFU) is a measure of the number of spores that were able, before or after storage, to germinate and form colonies. Table 1 shows the percentage of colony-forming units (in CFU/g) based on the starting value, as a measure of the viability rate or for the storage stability of the composition. The results shown are arithmetic averages from a triple determination.

TABLE 1
Storage stability of compositions
with spores of Trichoderma harzianum

| | | Proportion of germinable spores | | | |
|---|---|---|---|---|---|
| | | after storage at 40° C.:¹⁾ | | | |
| | | After 7 | After 14 | After 21 | After 28 |
| Composition | | days | days | days | days |
| Example a-2 | Example a-1 | 91% | 77% | 66% | 20% |
| | (80% by wt.) + | | | | |
| | T. harzianum | | | | |
| | spores | | | | |
| | (20% by wt.) | | | | |
| Example b-2 | Example b-1 | 94% | 89% | 62% | 41% |
| | (80% by wt.) + | | | | |
| | T. harzianum | | | | |
| | spores | | | | |
| | (20% by wt.) | | | | |
| Example c-2 | Example c-1 | 72% | 58% | 50% | 18% |
| | (80% by wt.) + | | | | |
| | T. harzianum | | | | |
| | spores | | | | |
| | (20% by wt.) | | | | |
| Comparative | T. harzianum | 73% | 30% | 7% | 2% |
| Example 1 | spores | | | | |
| Comparative | Commercially | 19% | 7% | 2% | 2% |
| Example 2 | available WP | | | | |
| | formulation of | | | | |
| | T. harzianum | | | | |

¹⁾expressed as the percentage of colony-forming units (in CFU/g) based on the starting value

The results show a distinct improvement in the compositions according to the invention over the pure spores (Comparative Example 1) and a commercial P formulation (Comparative Example 2).

Preparation of the Composition with Pseudomonas fluorescens

### Example b-3

Pseudomonas fluorescens was cultivated in anaerobic submerged fermentation on double-concentrated LB medium at 25° C. and pH 7 up to an optical density of 13. Thereafter, the bacteria biomass was harvested from the fermentation broth by centrifugation at 8000 g for 10 minutes. The cell pellet was then resuspended in a sodium chloride solution (0.9% by weight) and added to a suspension of Sipernat®50 and gum arabic. The resulting suspension contained about 8% by weight of silica, 7% by weight of gum arabic, 3% by weight of dry biomass and 81% by weight of water. The suspension was then spray-dried in a laboratory spray-dryer (Büchi B-290) at a gas inlet temperature of 72° C. The spray application was effected with a two-phase nozzle at an atomization pressure of about 1.35 bar. The flow rate of the drying air was 38 m³/h. The spray rate was about 5 ml/min. The parameters set led to an exit temperature of 52° C. and a residual moisture content of the product of 4-7% by weight of water. To produce the composition from polyethersiloxane b-1 and spray-dried biomass of Pseudomonas fluorescens, 1 g of dried vegetative Pseudomonas fluorescens cells was mixed into 9 g of the polyethersiloxane from Example b-1. The Example b-3 composition thus prepared was incubated at 40° C. for four weeks and the number of colony-forming units was determined immediately after the preparation (start value) and after 7, 14, 21 and 28 days. The procedure was the same with the comparative example. Table 2 shows the percentage of colony-forming units (in CFU/g) based on the start value, as a measure of the survival rate or of the storage stability of the composition. The results shown are arithmetic averages from a double determination.

TABLE 2
Storage stability of compositions comprising Pseudomonas fluorescens

| | | Proportion of vegetative cells | | | |
|---|---|---|---|---|---|
| | | after storage at 40° C:¹⁾ | | | |
| | | After 7 | After 14 | After 21 | After 28 |
| Composition | | days | days | days | days |
| Example b-3 | Example b-1 | 52% | 39% | 34% | 23% |
| | (90% by weight) + | | | | |
| | spray-dried | | | | |
| | biomass of | | | | |
| | Pseudomonas | | | | |
| | fluorescens | | | | |
| | (10% by weight) | | | | |
| Comparative | spray-dried biomass | 39% | 18% | 21% | 11% |
| example 3 | of Pseudomonas | | | | |
| | fluorescens | | | | |

¹⁾represented as percentage of colony-forming units (in CFU/g) based on the start. value

Even though Gram-negative bacterial cells such as Pseudomonas fluorescens are not known to be stable to heat or drying, the results show a clear improvement in the survival rate of Pseudomonas fluorescens as a constituent of the composition according to the invention compared to the spray-dried biomass on its own.

### Germination Test in the Presence of Adjuvants

With the aid of a germination test in the presence of 1.0% by weight of adjuvant, the effect of the siloxanes of formula (I) on the germinability of various commercial microorganisms was examined under application-relevant conditions. For this purpose, the commercial formulations were diluted with sterile aqueous adjuvant solution (1.0% by weight) in a decimal dilution series in a ratio of 1:100 000 to 1:1 000 000 000 and plated out on a suitable ready-made nutrient medium (Compact Dry from Nissui Pharmaceutical Co., Ltd.). Fungal spores were incubated at 25° C. for three days, bacterial spores at 30° C. for one day. Plates on which 10-100 CFU are visible were evaluated. The results summarized in Table 3 show the germination rate as a percentage of colony-forming units (in CFU/g) based on the value without adjuvants.

TABLE 3
Germination test for various commercial microorganisms

| | Germination rate in the presence of 1.0% | |
|---|---|---|
| | by wt. of siloxane of formula (I) ¹⁾ | |
| | | FZB24 ® WG |
| | Trichoderma | (Bacillus |
| Adjuvant | harzianurn | amyloliquefaciens) |
| Example a-1 | 118% | 124% |
| Example b-1 | 18% | 93% |
| Example c-1 | 97% | 100% |
| Break Thru ® S240 ²⁾ | 8% | 9% |

¹⁾ expressed as the percentage of colony-forming units (in CFU/g) based on the value without adjuvants
²⁾ commercially available polyether-modified trisiloxane, from Evonik

The inventive adjuvants a-1, b-1 and c-1 show distinctly improved germination rates over the comparative example Break Thru@ S240. They show improved biocompatibility.

It is also found that the siloxanes of formula (I) retain moisture for much longer than polyether-modified trisiloxanes and hence offer better conditions for microorganisms.

1. A composition, comprising:
at least one active microbiological ingredient, and
at least one siloxane of Formula (I)
M¹ₐM²_{b}D¹_{c}D²_{d} Formula (I)
wherein:
M¹=R¹₃SiO_{1/2};
M²=R¹₂R²₁SiO_{1/2};
D¹=R¹₂SiO_{2/2};
D²=R¹R²SiO_{2/2};
a=0 to 2;
b=0 to 2;
c=1.5 to 100;
d=0.5 to 25;
with the proviso that:
a+b=0 to 2; and
a+b+c+d≥4;
R¹ is in each case independently a monovalent hydrocarbyl radical;
R² is in each case independently a monovalent polyether radical.

2. The composition according to claim 1, wherein, as a further proviso:
c+d=2 to 125;
and/or
1≤c/d≤6.

3. The composition according to claim 1, wherein the at least one active microbiological ingredient is selected from the group consisting of microorganisms, organs of microorganisms and mixtures thereof.

4. The composition according to claim 1, wherein the at least one active microbiological ingredient has an antagonistic and/or hyperparasitic effect directed against a particular pathogen.

5. The composition according to claim 1, wherein the at least one active microbiological ingredient increases resistance and/or stress tolerance and/or nutrient availability in plants.

6. The composition according to claim 1, wherein the at least one active microbiological ingredient is selected from the group consisting of fungi, fungal organs and mixtures thereof.

7. The composition according to claim 1, wherein the at least one active microbiological ingredient is selected from the group consisting of Trichoderma spp. and its fungal organs and mixtures thereof.

8. The composition according to claim 1, wherein the at least one active microbiological agent comprises a fungal organ selected from the group consisting of spores, conidia, blastospores, chlamydospores, sclerotia, hyphal segments and mixtures thereof.

9. The composition according to claim 1, wherein the at least one active microbiological ingredient is or comprises a bacterium or a mixture of various bacteria.

10. The composition according to claim 1, wherein the at least one active microbiological ingredient comprises spores.

11. The composition according to claim 1, wherein the at least one active microbiological ingredient comprises vegetative cells.

12. The composition according to claim 1, wherein the at least one active microbiological ingredient comprises spores of Trichoderma harziamum and/or of Bacillus amyloliquefaciens and/or vegetative cells of Pseudomonas fluorescens.

13. The composition according to claim 1, wherein the at least one active microbiological ingredient is or comprises a virus or a mixture of various viruses.

14. A process for producing the composition according to claim 1, the process comprising:
mixing the at least one active microbiological ingredient and the at least one siloxane of Formula (I).

15. The process according to claim 14, wherein
i) the at least one active microorganism is cultivated;
ii) optionally the cultivated microorganism is processed by suitable separation, drying, grinding and/or dispersion methods; and
iii) the cultivated and optionally processed microorganism is suspended and/or dispersed in a siloxane of Formula (I).

16. The process according to claim 15, wherein the at least one microorganism and/or at least one organ of the microorganism, is processed ii) and after processing, is isolated by sieving, filtration, windsifting and/or centrifugation methods.

17. The process according to claim 14, further comprising reacting a composition comprising at least one SiH-functional siloxane of Formula (IV)
M¹ₐM³_{b}D¹_{c}D³_{d} Formula (IV)
wherein:
M¹=R¹₃SiO_{1/2};
M³=R¹₂HSiO_{1/2};
D¹=R¹₂SiO_{2/2};
D³=R¹HSiO_{2/2};
wherein:
the indices a, b, c and d are as defined for Formula (I);
R¹ is as defined for Formula (I);
in the manner of a hydrosilylation with at least one unsaturated, polyether, so as to obtain a composition comprising the at least one siloxane of Formula (I).

18. A method, comprising:
i) treating plants; or
ii) treating seeds; or
iii) treating soils; or
iv) producing as biostimulant; or
v) producing as probiotic food supplement and/or probiotic animal feed additive with the composition according to claim 1.

19. A probiotic medicament, comprising:
a composition according to claim 1.

20. A process for improving the storage stability of at least one active microbiological ingredient, the process comprising:
producing the composition of claim 1 by combining the at least one active microbiological ingredient with the at least one siloxane of Formula (I).
